(19) United States
(12) Patent Application Publication
(10) Pub. No.: US 2009/0216135 A1
(43) Pub. Date: Aug. 27, 2009

(54) Method, assembly, catheter, and processing device for obtaining an indication of cardiac output

(76) Inventor: Gheorghe Aurel Marie Pop, Nijimegen (NL)

Correspondence Address: MARK ZOVKO, 36504 28TH AVE S., FEDERAL WAY, WA, 98003, US

(21) Appl. No.: 12/322,808

(22) Filed: Feb. 6, 2009

(30) Foreign Application Priority Data
Feb. 7, 2008 (NL) NL 1035002

(51) Int. Cl. A61B 5/02

(52) U.S. Cl. 600/505

(57) ABSTRACT

The invention relates to a method for obtaining an indication of cardiac output, comprising the steps of introducing into the bloodstream a liquid with a temperature lower than the temperature of the blood, measuring downstream the temperature variation of the blood, and performing a thermodilution algorithm on the basis of the measured temperature variation of the blood. The invention is distinguished in that the temperature variation is measured upstream of the heart in the blood flow of a vein in which the flow rate is substantially proportional to the cardiac output, and the flow rate in the vein is determined using the thermodilution algorithm. The invention also relates to an assembly, catheter and processing device for obtaining an indication of cardiac output.

The invention relates to a method for obtaining an indication of cardiac output, comprising the steps of introducing into the bloodstream a liquid with a temperature lower than the temperature of the blood, measuring downstream the temperature variation of the blood and performing a thermodilution algorithm on the basis of the measured temperature variation of the blood.

Such a method for obtaining an indication of cardiac output is known as Swan-Ganz catheterization. In this known method the distal end of a so-called Swan-Ganz catheter is introduced successively via the superior vena cava, the right atrium and the right ventricle into the pulmonary artery (arteria pulmonalis). In this position a liquid is introduced into the bloodstream in the right atrium via a lumen of the Swan-Ganz catheter. This liquid has a temperature which is lower than the temperature of the blood, so that the temperature of the blood undergoes a change due to the addition of the liquid. The blood with the liquid therein then flows out of the right atrium via the right ventricle into the pulmonary artery. The temperature variation of the blood is here measured by means of a thermistor arranged close to the distal end of the Swan-Ganz catheter on the wall thereof. On the basis of this measured temperature variation the cardiac output is then determined by performing a so-called thermodilution algorithm.

When inserted, the Swan-Ganz catheter covers a path through the heart wherein it passes through, among others, the tricuspid valve (valvula tricuspidalis) and the pulmonary valve (valva trunci pulmonalis) and also takes a sharp bend in the right ventricle. The drawback of Swan-Ganz catheterization is that the tip of the catheter can herein cause damage to the heart. When taking the bend in the right ventricle, the tip of the catheter can for instance touch a wall of the heart, whereby serious ventricular arrhythmia can occur.

The invention has for its object to obviate or at least alleviate this.

The invention is distinguished for this purpose in that the temperature variation is measured upstream of the heart in the blood flow of a vein in which the flow rate is substantially proportional to the cardiac output, and the flow rate in the vein is determined using the thermodilution algorithm.

These measures make it possible to obtain an indication of cardiac output without a catheter being introduced into the pulmonary artery through the tricuspid valve and the pulmonary valve via the right ventricle, as for instance in Swan-Ganz catheterization. This has the advantage of considerably reducing the risk of damage to the heart through introduction of the catheter.

In a favourable embodiment of the method according to the invention the vein is a vena cava. The flow rate in the superior vena cava and the flow rate in the inferior vena cava are found to be substantially proportional to the cardiac output and the flow rate in each of these venae cavae determined with the thermodilution algorithm is found to be a representative indication of cardiac output. A catheter can moreover be introduced into the superior vena cava in relatively simple manner, for instance via the jugular vein (vena jugularis) or subclavian vein (vena subclavia), or even via the vein in one of the arms (vena brachialis), or into the inferior vena cava via the femoral vein (vena femoralis). In a further embodiment hereof, the temperature variation is measured close to the end of the vena cava. This measure makes it possible for the distance along the vena cava between the location where the liquid is introduced into the bloodstream and the location where the temperature variation of the blood is measured to be as large as possible. This has the advantage that a better mixing of the liquid and the blood is then obtained, whereby the indication of cardiac output can be more accurate.

In a further embodiment of the method according to the invention the distal end of a catheter is introduced via the vena cava into the right atrium, the liquid is introduced into the blood via a lumen of the catheter which debouches into an outflow opening in a wall of the catheter, and the temperature variation of the blood is measured by means of a thermistor located more distally on the wall of the catheter relative to the outflow opening. These steps enable a precise indication of cardiac output with a catheter of simple design.

In a further embodiment of the method according to the invention the trend of the flow rate in the vein is determined. The trend of the flow rate in a vein upstream of the heart in which the flow rate is substantially proportional to the cardiac output, such as the flow rate in one of the venae cavae, is found in practice to represent a useful indication of cardiac output. A sudden sharp fall in the flow rate in the vein is for instance an indication that the cardiac output has fallen sharply. The medication or other treatment for the heart can then be modified to the new situation.

In a further embodiment of the method according to the invention the flow rate in the vein is multiplied by a correction factor representing the ratio of the cardiac output and the flow rate in the vein. In this way an absolute value can be obtained as indication of cardiac output. This method of determining an indication of the absolute value of the cardiac output is possible by making use of the insight that the ratio of the cardiac output and the flow rate in the vein remains substantially the same in a determined period. In a further embodiment hereof, the vein is the superior vena cava and the correction factor lies in a range between substantially 1.4 and substantially 1.7. It has been found that a correction factor lying within this range is representative of the ratio of the cardiac output and the flow rate in the superior vena cava. In an alternative embodiment hereof, the vein is the inferior vena cava and the correction factor lies in a range between substantially 2.0 and substantially 2.5. It has been found that a correction factor lying within this range is representative of the ratio of the cardiac output and the flow rate in the inferior vena cava. In an alternative embodiment hereof, the method comprises the steps of determining the cardiac output by means of another method simultaneously with determining of the flow rate in the vein, and determining the correction factor by dividing the determined cardiac output by the flow rate determined in the vein. It is for instance possible in this way, via a for instance expensive but accurate method or a more accurate method with a greater risk of damage to the heart, to make a once-only determination of the cardiac output and to then obtain a continuous indication of the cardiac output by multiplying the flow rate in the vein by the found correction factor. This has the advantage for instance that it is not necessary to determine the cardiac output continuously by means of the expensive method or the method with a greater risk of damage to the heart.

The invention also relates to an assembly for obtaining an indication of cardiac output, comprising a catheter comprising a tubular body with a proximal end and a distal end, a thermistor on the wall of the catheter close to the distal end thereof, a conductor connected to the thermistor and extending therefrom through the tubular body to the proximal end, an outflow opening in a wall of the catheter between the proximal end of the catheter and the thermistor, and a lumen connected to the outflow opening and extending therefrom through the tubular body to the proximal end, a processing device which is connected at the proximal end to the conductor and which is adapted to measure a temperature variation by means of the thermistor and to perform a thermodilution algorithm on the basis of the measured temperature variation, wherein the distance along the tubular body between the outflow opening and the thermistor lies in a range between substantially 10 centimetres and substantially 18 centimetres—such as 11, 12, 13, 14, 15, 16, 17 centimetres—, the thermistor is arranged close to the distal end of the catheter and the flow rate in a vein is determined with the thermodilution algorithm.

Using this assembly it is possible to position the thermistor close to the end of the vena cava for the purpose of measuring the temperature variation of the blood, while a liquid can be introduced into the bloodstream and at sufficient distance from the thermistor that a good mixing of the liquid with the blood is realized for the purpose of an accurate determination of the flow rate in the vena cava. This has the advantage that an indication of the cardiac output can be obtained without a catheter being introduced into the pulmonary artery through the tricuspid valve and the pulmonary valve via the right ventricle, and that the risk of damage to the heart due to introduction of the catheter is reduced considerably compared to for instance the use of a Swan-Ganz catheter. The feature that the distance along the tubular body between the outflow opening and the thermistor lies in a range between substantially 10 centimetres and substantially 18 centimetres—such as 11, 12, 13, 14, 15, 16, 17 centimetres—is particularly favourable when the catheter is introduced into the superior vena cava via an opening in the jugular vein (vena jugularis) or subclavian vein (vena subclavia). The outflow opening is then still situated in the body while the thermistor can be situated close to the end of the vena cava.

In a further embodiment of the assembly according to the invention the processing device is adapted to determine the trend of the flow rate in the vein. The trend of the flow rate in for instance the vena cava is found in practice to represent a useful indication of the cardiac output. Determining and displaying the trend of the flow rate in the vena cava, and thereby the trend of the cardiac output, by the processing device assists the physician in the treatment of a patient.

In a further embodiment of the assembly according to the invention the processing device is adapted to multiply the flow rate determined in the vein by a correction factor representing the ratio of the cardiac output and the flow rate in the vein. This measure makes it possible for instance to display on the processing device an absolute value as indication of the cardiac output.

In a further embodiment hereof, the vein is the superior vena cava and the correction factor lies in a range between substantially 1.4 and substantially 1.7. It has been found that a correction factor lying within this range is representative of the ratio of the cardiac output and the flow rate in the superior vena cava. In an alternative embodiment hereof, the vein is the inferior vena cava and the correction factor lies in a range between substantially 2.0 and substantially 2.5. It has been found that a correction factor lying within this range is representative of the ratio of the cardiac output and the flow rate in the inferior vena cava. In a further alternative embodiment hereof, the processing device comprises setting means with which the correction factor can be set. This measure makes it possible to modify the correction factor to the specific conditions. This has the advantage that determination of the cardiac output can be more accurate.

In a further embodiment of the assembly according to the invention the distance along the catheter between the thermistor and the distal end of the catheter is less than substantially 4 centimetres, such as 3, 2, 1 centimetres. This measure makes it for instance possible for other sensors to be arranged in the possible space between the distal end of the catheter and the thermistor, with which sensors measurements can then be performed in the right atrium while the thermistor is situated close to the end of the vena cava and the end of the catheter is still situated upstream of the tricuspid valve.

The invention also relates to a catheter for forming an assembly, comprising a tubular body with a proximal and a distal end, a thermistor on the wall of the catheter close to the distal end thereof, a conductor connected to the thermistor and extending therefrom through the tubular body to the proximal end, an outflow opening in a wall of the catheter between the proximal end of the catheter and the thermistor, and a lumen connected to the outflow opening and extending therefrom through the tubular body to the proximal end, wherein the distance along the tubular body between the outflow opening and the thermistor lies in a range between substantially 10 centimetres and substantially 18 centimetres—such as 11, 12, 13, 14, 15, 16, 17 centimetres—and the thermistor is arranged close to the distal end of the catheter.

In a further embodiment of the catheter according to the invention the distance along the catheter between the thermistor and the distal end of the catheter is less than substantially 4 centimetres, such as 3, 2, 1 centimetres.

The invention also relates to a processing device for forming an assembly, comprising connecting means for connecting the device to a thermistor, this processing device being adapted to measure a temperature variation by means of the thermistor and to perform a thermodilution algorithm on the basis of the measured temperature variation,

wherein the flow rate in a vein is determined with the thermodilution algorithm, after which the cardiac output is determined by multiplication by a correction factor representing the ratio of the cardiac output and the flow rate in a vein.

In a further embodiment of a processing device according to the invention the vein is the superior vena cava and the correction factor lies in a range between substantially 1.4 and substantially 1.7. In an alternative embodiment hereof, the vein is the inferior vena cava and the correction factor lies in a range between substantially 2.0 and substantially 2.5. In a further alternative embodiment hereof, the processing device comprises setting means with which the correction factor can be set.

The present invention will be further elucidated hereinbelow on the basis of an exemplary embodiment as shown in the accompanying figures. This is a non-limitative exemplary embodiment. In the figures:

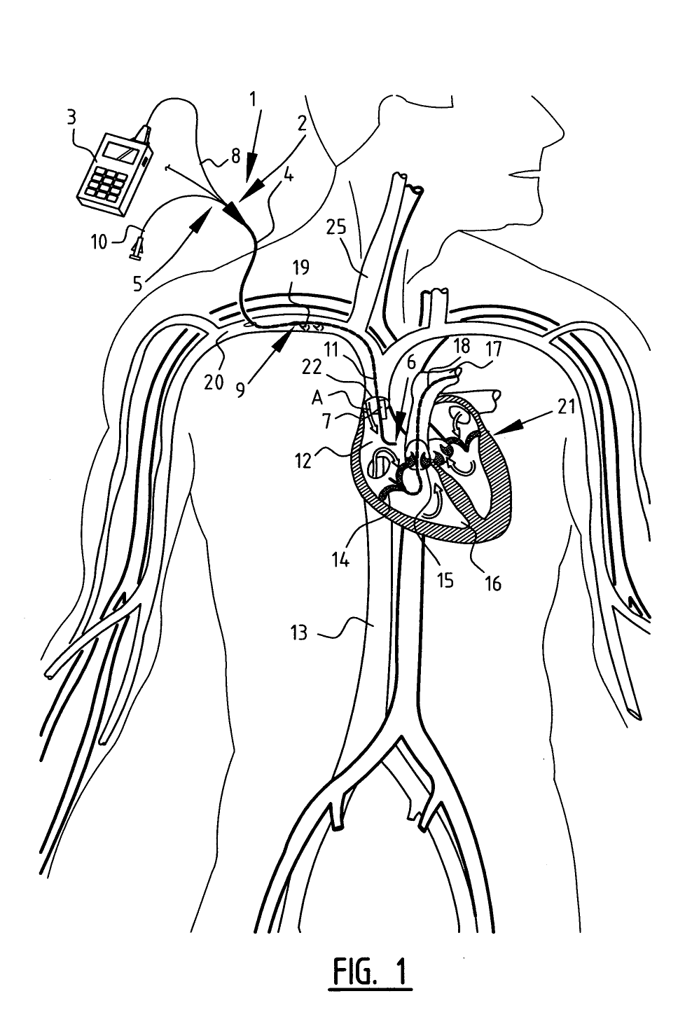

FIG. 1 is a schematic representation of a heart and a part of the bloodstream, wherein a-catheter according to the invention is positioned in the superior vena cava for the purpose of obtaining an indication of the cardiac output; and

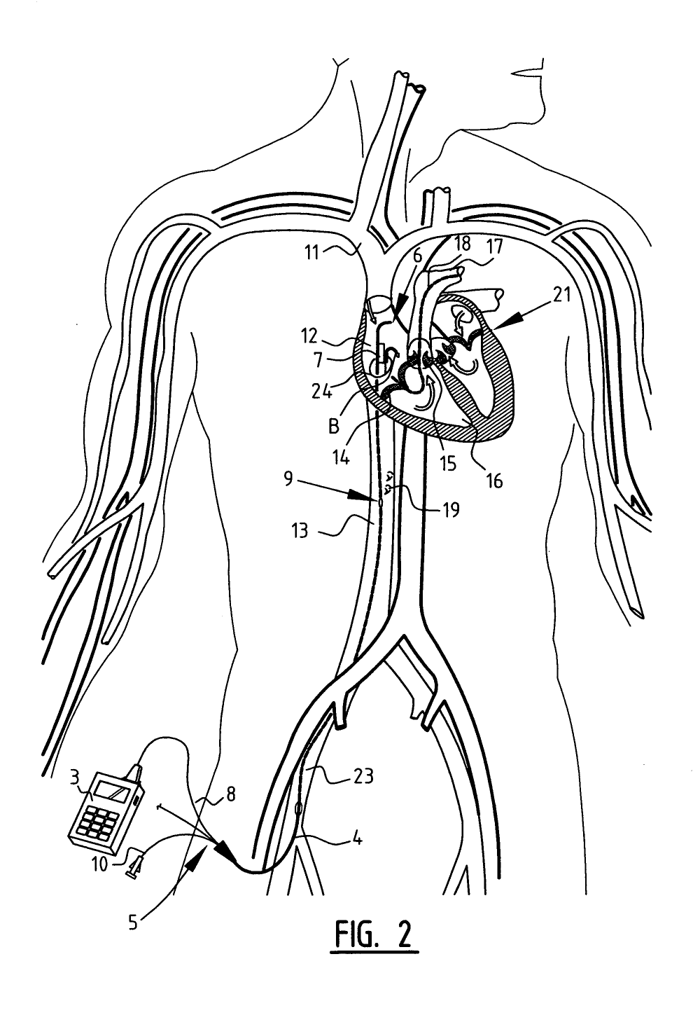

FIG. 2 is a schematic representation of a heart and a part of the bloodstream, wherein a catheter according to the invention is positioned in the inferior vena cava for the purpose of obtaining an indication of the cardiac output.

FIGS. 1 and 2 show an assembly 1 for determining cardiac output during use. Assembly 1 is shown with a catheter 2 and a processing device 3. Catheter 2 is shown with a tubular body 4 with a proximal end 5 and a distal end 6. A thermistor 7 is shown schematically on the wall of catheter 2 close to the distal end 6 thereof. Thermistor 7 is connected to a conductor 8 extending therefrom through tubular body 4 to the proximal end 5. Conductor 8 is connected at proximal end 5 to processing device 3. Between proximal end 5 of catheter 2 and thermistor 7 the catheter 2 has in the wall thereof an outflow opening 9 which is connected to a lumen to extending therefrom through tubular body 4 to proximal end 5.

FIG. 1 shows that in the position of use of assembly 1 a part of tubular body 4 of catheter 2 is situated in the subclavian vein 20 and in the superior vena cava 11. The blood flow in the superior vena cava 11 is indicated by means of arrow A. The superior vena cava lies upstream of heart 21. Distal end 6 is situated in the right atrium 12. Thermistor 7 is situated close to the end 22 of the superior vena cava 11 in the blood flow A of the superior vena cava 11. During use the distal end 6 of the catheter is not introduced into the pulmonary artery 17 through tricuspid valve 14 and pulmonary valve 15 via the right ventricle 16, as shown by the broken line 18.

During use a liquid 19 is introduced into the blood at the position of subclavian vein 20 by means of a pump means (not shown) via lumen 10 and through outflow opening 9. This liquid 19 then has a temperature lower than the temperature of the blood. The temperature of the blood undergoes a change due to the addition of the liquid. The blood with the liquid therein then flows to the right atrium 12. By means of thermistor 7 the temperature variation of the blood in blood flow A is measured through time close to the end 22 of the superior vena cava 11 by processing device 3. Processing device 3 is adapted to measure a temperature variation by means of thermistor 7 and to perform a thermodilution algorithm on the basis of the measured temperature variation. The flow rate in the superior vena cava 11 is determined with the thermodilution algorithm. Because a part of the cardiac output flows into the right atrium 12 via the superior vena cava 11 and the rest of the cardiac output flows into the right atrium 12 via the inferior vena cava 13, in order to determine an absolute value as indication of the cardiac output the flow rate determined in the superior vena cava 11 is multiplied by a correction factor by processing device 3, wherein this correction factor represents the ratio of the cardiac output and the flow rate in the superior vena cava. It has been found that the ratio of the cardiac output and the flow rate in the superior vena cava 11 lies within a range between substantially 1.4 and substantially 1.7. In the shown embodiment the correction factor can be set in processing device 3 by means of setting means, formed for instance by the keyboard and a control which is connected thereto and with which the performed thermodilution algorithm is controlled.

In FIG. 1 the distance along tubular body 4 between outflow opening 9 and thermistor 7 is such that thermistor 7 is positioned close to the end 22 of the superior vena cava 11 while the liquid 19 can be introduced into the bloodstream at sufficient distance from thermistor 7 that a good mixing of the liquid with the blood is realized for the purpose of an accurate determination of the flow rate in the superior vena cava 11. A distance along tubular body 4 between outflow opening 9 and thermistor 7 in the range between substantially 10 centimetres and substantially 18 centimetres—such as 11, 12, 13, 14, 15, 16, 17 centimetres—has been found favourable. The distance along tubular body 4 between thermistor 7 and the distal end 6 of catheter 2 is such that distal end 22 of catheter 2 is situated upstream of the tricuspid valve 14. Other sensors, with which measurements can be performed in the right atrium 12, can be arranged in the space between distal end 6 of catheter 22 and the thermistor 7.

FIG. 2 shows that in the position of use of assembly 1 a part of tubular body 4 of catheter 2 is situated in the femoral vein 23 and in the inferior vena cava 13. The blood flow in the inferior vena cava 13 is shown by means of arrow B. The inferior vena cava 13 lies upstream of heart 21. Distal end 6 is situated in the right atrium 12. Thermistor 7 is situated close to the end 24 of the inferior vena cava 13 upstream of the end 24 of the superior vena cava 11. During use the distal end 6 of the catheter is not introduced into pulmonary artery 17 through tricuspid valve 14 and pulmonary valve 15 via the right ventricle 16, as shown by the broken line 18.

During use a liquid 19 is introduced into the blood at the position of the inferior vena cava 13 by means of a pump means (not shown) via lumen 10 and through outflow opening 9. This liquid 19 then has a temperature lower than the temperature of the blood. The temperature of the blood undergoes a change due to the addition of the liquid. The blood with the liquid therein then flows to the right atrium 12. By means of thermistor 7 the temperature variation of the blood in blood flow B is measured through time close to the end 24 of the inferior vena cava 13 by processing device 3. Processing device 3 is adapted to perform a thermodilution algorithm on the basis of the measured temperature variation. The flow rate in the inferior vena cava 13 is determined with the thermodilution algorithm. Because a part of the cardiac output flows into the right atrium 12 via the inferior vena cava 13 and the rest of the cardiac output flows into the right atrium 12 via the superior vena cava 11, in order to determine an absolute value as indication of the cardiac output the flow rate determined in the inferior vena cava 13 is multiplied by a correction factor by processing device 3, wherein this correction factor represents the ratio of the cardiac output and the flow rate in the inferior vena cava. It has been found that the ratio of the cardiac output and the flow rate in the inferior vena cava 13 lies within a range between substantially 2.0 and substantially 2.5. In the shown embodiment the correction factor can be set in processing device 3 by means of setting means, formed for instance by the keyboard and a control which is connected thereto and with which the performed thermodilution algorithm is controlled.

It will be apparent to the skilled person that, instead of multiplying the flow rate in a vena cava by a correction factor representing the ratio of the cardiac output and the flow rate in the vena cava, the flow rate in the vena cava can also be divided by the inverse of this correction factor.

FIG. 1 shows that in the position of use of assembly 1 a part of tubular body 4 of catheter 2 is situated in the subclavian vein 20 and in the superior vena cava 11. Tubular body 4 of catheter 2 can also be introduced into the superior vena cava 11 via the jugular vein 25, and a part of tubular body 4 of catheter 2 is situated in jugular vein 25 instead of in subclavian vein 20. During use the liquid 19 is then introduced into the bloodstream in the jugular vein. Tubular body 4 of catheter can also be introduced into the superior vena cava 11 via the vein in one of the arms (vena brachialis), and a part of tubular body 4 of catheter 2 is situated in the vein in one of the arms as well as in subclavian vein 20.

Shown in the figures is that the distal end 6 of catheter 2 is situated at a distance from thermistor 7 such that in the shown position of use the distal end 6 is situated in the middle of the right atrium 12. This distance can also be smaller so that for instance the distal end 6 of catheter 2 is situated closer to thermistor 7, for instance against it.

1. Method for obtaining an indication of cardiac output, comprising the steps of:
introducing into the bloodstream a liquid with a temperature lower than the temperature of the blood;
measuring downstream the temperature variation of the blood; and
performing a thermodilution algorithm on the basis of the measured temperature variation of the blood,
characterized in that
the temperature variation is measured upstream of the heart in the blood flow of a vein in which the flow rate is substantially proportional to the cardiac output; and
the flow rate in the vein is determined using the thermodilution algorithm.

2. Method as claimed in claim 1, wherein
the vein is a vena cava.

3. Method as claimed in claim 2, wherein
the temperature variation is measured close to the end of the vena cava.

4. Method as claimed in claim 3, wherein
the distal end of a catheter is introduced via the vena cava into the right atrium,
the liquid is introduced into the blood via a lumen of the catheter which debouches into an outflow opening in a wall of the catheter; and
the temperature variation of the blood is measured by means of a thermistor located more distally on the wall of the catheter relative to the outflow opening.

5. Method as claimed in any of the foregoing claims, wherein
the trend of the flow rate in the vein is determined.

6. Method as claimed in any of the foregoing claims, wherein
the flow rate in the vein is multiplied by a correction factor representing the ratio of the cardiac output and the flow rate in the vein.

7. Method as claimed in claim 6, wherein
the vein is the superior vena cava; and
the correction factor lies in a range between substantially 1.4 and substantially 1.7.

8. Method as claimed in claim 6, wherein
the vein is the inferior vena cava; and
the correction factor lies in a range between substantially 2.0 and substantially 2.5.

9. Method as claimed in claim 6, characterized by the steps of:
determining the cardiac output by means of another method simultaneously with determining of the flow rate in the vein; and
determining the correction factor by dividing the determined cardiac output by the flow rate determined in the vein.

10. Assembly for obtaining an indication of cardiac output, comprising:
a catheter comprising:
a tubular body with a proximal end and a distal end;
a thermistor on the wall of the catheter close to the distal end thereof;
a conductor connected to the thermistor and extending therefrom through the tubular body to the proximal end;
an outflow opening in a wall of the catheter between the proximal end of the catheter and the thermistor; and
a lumen connected to the outflow opening and extending therefrom through the tubular body to the proximal end;
a processing device which is connected at the proximal end to the conductor and which is adapted to measure a temperature variation by means of the thermistor and to perform a thermodilution algorithm on the basis of the measured temperature variation,
wherein
the distance along the tubular body between the outflow opening and the thermistor lies in a range between substantially 10 centimetres and substantially 18 centimetres;
the thermistor is arranged close to the distal end of the catheter; and
the processing device is adapted to determine the cardiac output on the basis of a flow rate in a vein determined by means of the thermodilution algorithm.

11. Assembly as claimed in claim 10,
characterized in that
the processing device is adapted to determine the trend of the flow rate determined in the vein.

12. Assembly as claimed in either of the claims 10 and 11,
characterized in that
the processing device is adapted to multiply the flow rate determined in the vein by a correction factor representing the ratio of the cardiac output and the flow rate determined in the vein.

13. Assembly as claimed in claim 12,
characterized in that
the flow rate determined in a vein is the flow rate in the superior vena cava; and
the correction factor lies in a range between substantially 1.4 and substantially 1.7.

14. Assembly as claimed in claim 12,
characterized in that
the flow rate determined in a vein is the flow rate in the inferior vena cava; and
the correction factor lies in a range between substantially 2.0 and substantially 2.5.

15. Assembly as claimed in any of the claims 12-14,
characterized in that
the processing device comprises setting means with which the correction factor can be set.

16. Assembly as claimed in any of the claims 12-15,
characterized in that
the distance along the catheter between the thermistor and the distal end of the catheter is less than substantially 4 centimetres.

17. Catheter for forming an assembly as claimed in any of the claims 10-14, comprising:
a tubular body with a proximal and a distal end;
a thermistor on the wall of the catheter close to the distal end thereof;
a conductor connected to the thermistor and extending therefrom through the tubular body to the proximal end;
an outflow opening in a wall of the catheter between the proximal end of the catheter and the thermistor; and
a lumen connected to the outflow opening and extending therefrom through the tubular body to the proximal end;
wherein
the distance along the tubular body between the outflow opening and the thermistor lies in a range between substantially 10 centimetres and substantially 18 centimetres; and
the thermistor is arranged close to the distal end of the catheter.

18. Catheter as claimed in claim 17,
characterized in that
the distance along the catheter between the thermistor and the distal end of the catheter is less than substantially 4 centimetres.

19. Processing device for forming an assembly as claimed in any of the claims 10-16, comprising:
connecting means for connecting the device to a thermistor; this processing device being adapted to measure a temperature variation by means of the thermistor and to perform a thermodilution algorithm on the basis of the measured temperature variation,
wherein
the processing device is further adapted to multiply a flow rate in a vein determined by means of the thermodilution algorithm by a correction factor representing the ratio of the cardiac output and the flow rate determined in a vein.

20. Processing device as claimed in claim 19,
characterized in that
the flow rate determined in a vein is the flow rate in the superior vena cava; and
the correction factor lies in a range between substantially 1.4 and substantially 1.7.

21. Processing device as claimed in claim 19,
characterized in that
the flow rate determined in a vein is the flow rate in the inferior vena cava; and
the correction factor lies in a range between substantially 2.0 and substantially 2.5.

22. Processing device as claimed in any of the claims 19-21,
characterized in that
the processing device comprises setting means with which the correction factor can be set.
